(19) United States
(12) Patent Application Publication
(10) Pub. No.: US 2004/0192595 A1
(43) Pub. Date: Sep. 30, 2004

(54) Treatment of lung disorders

(76) Inventors: Kazunori Murakami, Galveston, TX (US); Perenlei Enkhbaatar, Galveston, TX (US); Lillian D. Traber, Galveston, TX (US); Donald S. Prough, Galveston, TX (US); David N. Herndon, Galveston, TX (US); Daniel L. Traber, Galveston, TX (US)

Correspondence Address: GTC BIOTHERAPEUTICS, INC., 175 CROSSING BOULEVARD, SUITE 410, FRAMINGHAM, MA, 01702, US

(21) Appl. No.: 10/396,855

(22) Filed: Mar. 25, 2003

(51) Int. Cl. A61K 38/48

(52) U.S. Cl. 514/12

(57) ABSTRACT

The invention features methods of treating a subject having a lung disorder such as lung inflammation and injury, by administering antithrombin III by inhalation.

### SUMMARY OF THE INVENTION

The invention is based, in part, on the discovery that aerosolized antithrombin III (ATIII) is effective in treating lung disorders, e.g., lung inflammation and injury. It was found that lower doses of aerosolized ATIII were more effective at treating acute septic lung injury than higher doses of intravenously administered ATIII. Thus, administration of ATIII by inhalation provides more efficient treatment of lung disorders, e.g., lung inflammation and injury, than intravenous administration.

Accordingly, in one aspect, the invention features a method of treating a subject having a lung disorder, e.g., lung inflammation and/or injury, which includes administration of a therapeutically effective amount of ATIII by inhalation. The lung disorder can be an acute or chronic lung disorder. In one embodiment, the lung disorder is an acute lung injury, e.g., septic acute lung injury or acute respiratory distress syndrome (ARDS). Lung injury and/or inflammation can be in response to, e.g., exposure to an external agent, e.g., a viral agent (e.g., Pseudomonas pneumonia), smoke or asbestos. In other embodiments, the lung disorder can be, e.g., lung or pleural neoplasia, interstitial lung disease and/or organizing pleuitis.

In one embodiment, the ATIII is administered using a jet aerosol or ultrasonic nebulizer system, or by a dry powder inhalation system. Such systems for aerosol administration are known.

In one embodiment, the ATIII is human ATIII. The ATIII can be naturally derived, e.g., from plasma, or recombinantly produced. Plasma derived ATIII is commercially available. In a preferred embodiment, the anti-thrombin III is transgenically produced, e.g., the ATIII is obtained from milk from a transgenic diary animal, e.g., a cow, a goat, a rabbit, or a mouse. Methods of producing ATIII in the milk of a transgenic animal are described in U.S. Pat. No. 5,843,705, the contents of which is incorporated herein by reference.

In a preferred embodiment, the subject is administered an aerosol composition that includes ATIII and a pharmaceutically acceptable carrier. Examples of pharmaceutically acceptable carriers include water and saline.

In one embodiment, the subject is periodically administered ATIII by inhalation, e.g., the subject is administered ATIII at regular intervals. For example, the subject can be administered aerosol ATIII at the onset of lung inflammation and/or injury and then at set intervals after the initial administration, e.g., ATIII can be administered by inhalation every hour, 2 hours, 3 hours, 4 hours, 6 hours, twice a day, or three, four, five, six time a day. The period of administration can be over a period of about 24, 48, 72, 96, 120, 144 or 168 hours. In another embodiment, the subject is administered ATIII by inhalation as needed, e.g., ATIII is administered upon indication of one or more continued or reoccurring symptom(s) of lung inflammation or injury.

An effective dose of ATIII, e.g., transgenically produced ATIII, can be between about 10-300 U/kg, 25-125 U/kg, 50-100 U/kg, or 60-75 U/kg of body weight. In another aspect, an effective dose can be greater than about 1 mg/kg, 5 mg/kg, 10 mg/kg, but less than about 150 mg/kg, 100 mg/kg, 70 mg/kg.

In a preferred embodiment, the dose of aerosol ATIII used is less than 10%, 20%, 30%, 40%, 50%, 60% the dose of ATIII intravenously administered to treat the same disorder, e.g., to have the same effect on one or more symptom of lung inflammation or injury.

In another embodiment, the invention features a kit for treating lung disorders. Preferably, the kit includes a therapeutically effective amount of an aerosol form of ATIII, and instructions for use. Preferably, the aerosol further includes a pharmaceutically acceptable carrier. Examples of pharmaceutically acceptable carriers include water and saline.

In one embodiment, an effective dose of ATIII, e.g., transgenically produced ATIII, can be between about 10-300 U/kg, 25-125 U/kg, 50-100 U/kg, or 60-75 U/kg of body weight. In another aspect, an effective dose can be greater than about 1 mg/kg, 5 mg/kg, 10 mg/kg, but less than about 150 mg/kg, 100 mg/kg, 70 mg/kg.

In a preferred embodiment, the kit is a kit for treating an acute or chronic lung disorder. Preferably, the lung disorder is an acute lung injury, e.g., septic acute lung injury or acute respiratory distress syndrome (ARDS). Lung injury and/or inflammation can be in response to, e.g., exposure to an external agent, e.g., a viral agent (e.g., Pseudomonas pneumonia), smoke or asbestos. In other embodiments, the lung disorder can be, e.g., lung or pleural neoplasia, interstitial lung disease and/or organizing pleuitis.

In one embodiment, the kit includes ATIII in a jet aerosol or ultrasonic nebulizer system, or a dry powder inhalation system.

In one embodiment, the kit includes an aerosol form of human ATIII. The ATIII can be naturally derived, e.g., from plasma, or recombinantly produced. In a preferred embodiment, the anti-thrombin III is transgenically produced, e.g., the ATIII is obtained from milk from a transgenic diary animal, e.g., a cow, a goat, a rabbit, or a mouse.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph depicting the effect of administering nebulized ATTII on pulmonary gas exchange (PaO2/FiO2 ratio) in a sheep model having sepsis due to smoke inhalation.

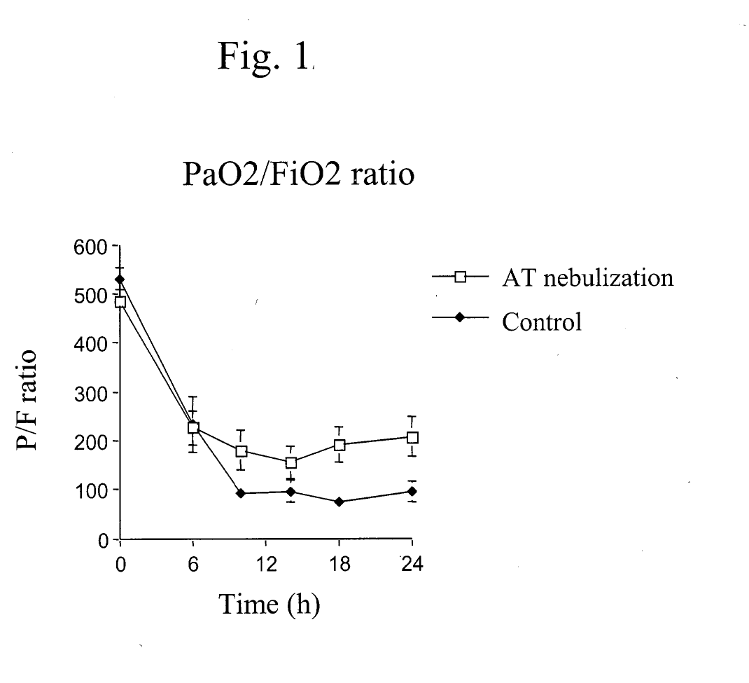

FIG. 2 is a graph depicting the effect of administering nebulized ATIII on pulmonary shunt fraction in a sheep model having sepsis due to smoke inhalation.

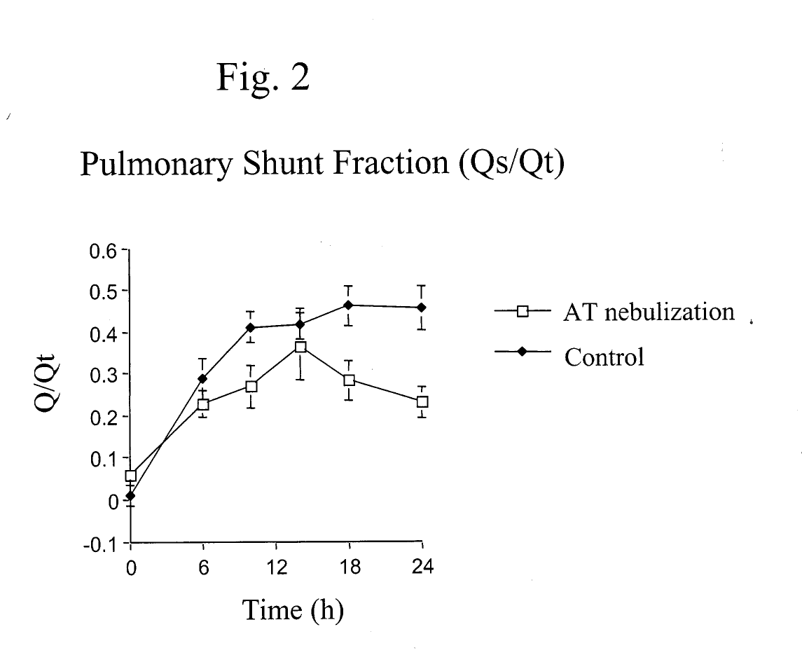

FIG. 3 is a graph depicting the effect of administering nebulized ATIII on mean artial pressure in a sheep model having sepsis due to smoke inhalation.

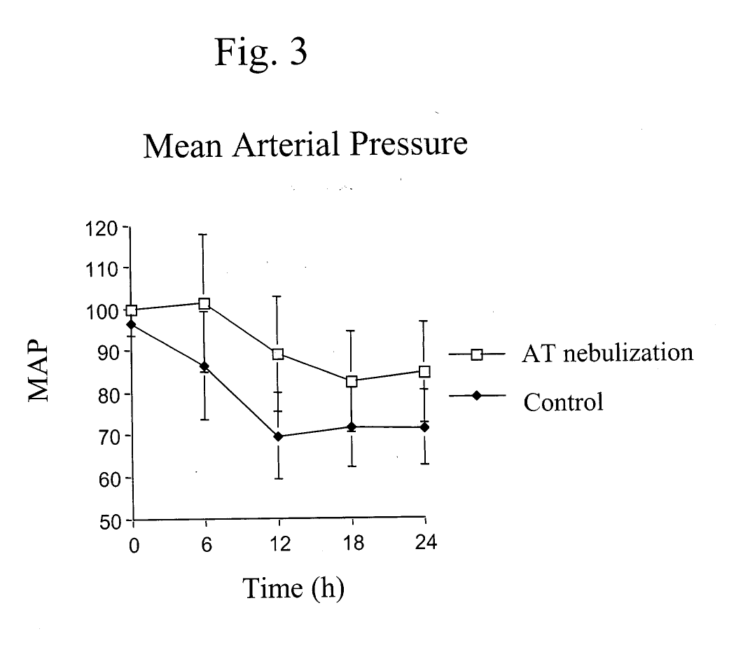

FIG. 4 is a graph depicting the effect of administering nebulized ATIII on left strium pressure in a sheep model having sepsis due to smoke inhalation.

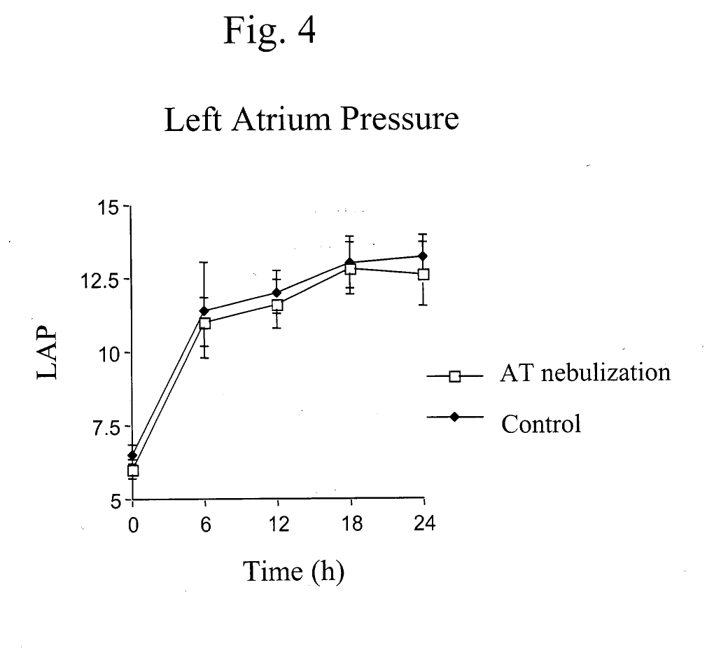

FIG. 5 is a graph depicting the effect of administering nebulized ATIII on pulmonary artery pressure in a sheep model having sepsis due to smoke inhalation.

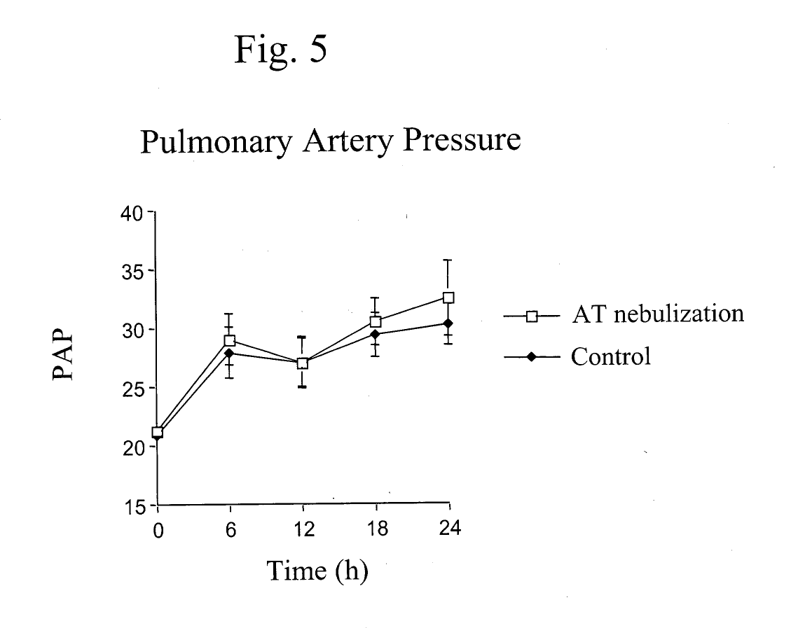

FIG. 6 is a graph depicting the effect of administering nebulized ATIII on cardiac index in a sheep model having sepsis due to smoke inhalation.

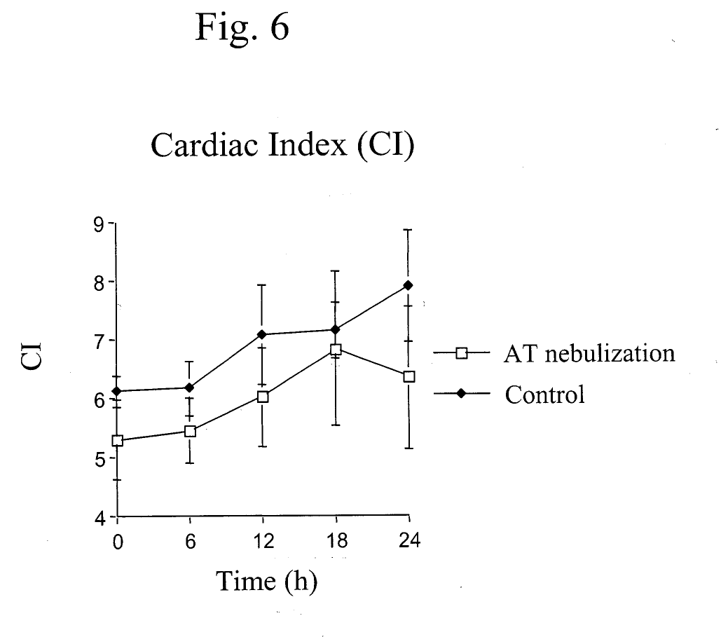

FIG. 7 is a graph depicting the effect of administering nebulized ATIII on left ventricular stroke work index (LVSWI) in a sheep model having sepsis due to smoke inhalation.

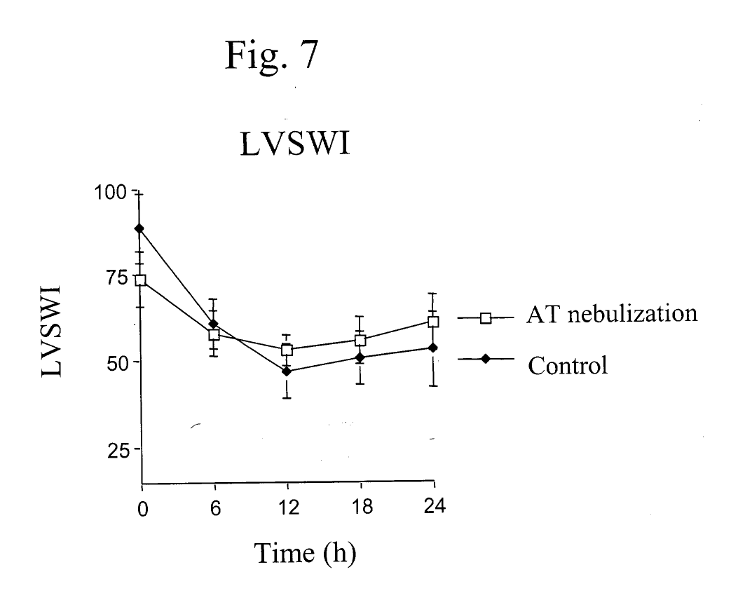

FIG. 8 is a graph depicting the effect of administering nebulized ATIII on body temperature in a sheep model having sepsis due to smoke inhalation.

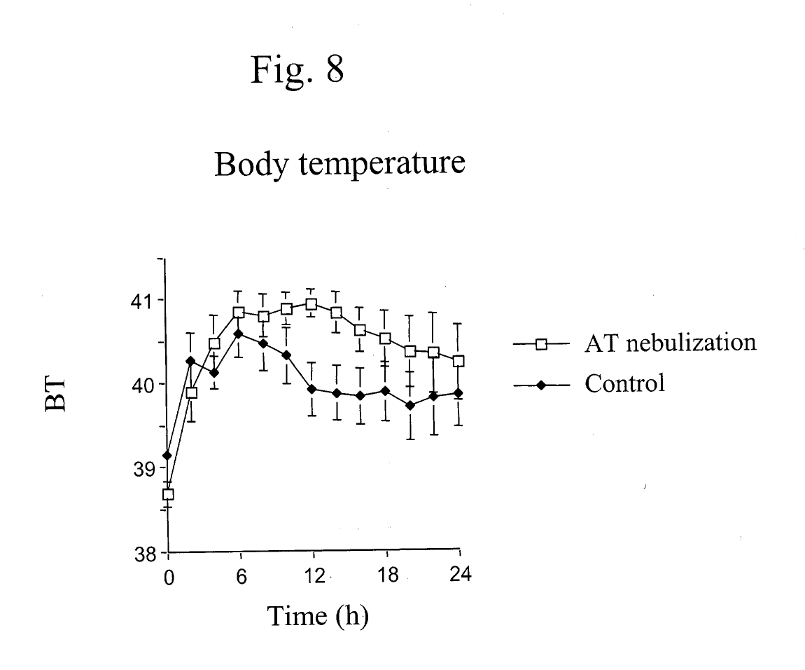

FIG. 9 is a graph depicting the effect of administering nebulized ATIII on left plasma NOx levels in a sheep model having sepsis due to smoke inhalation.

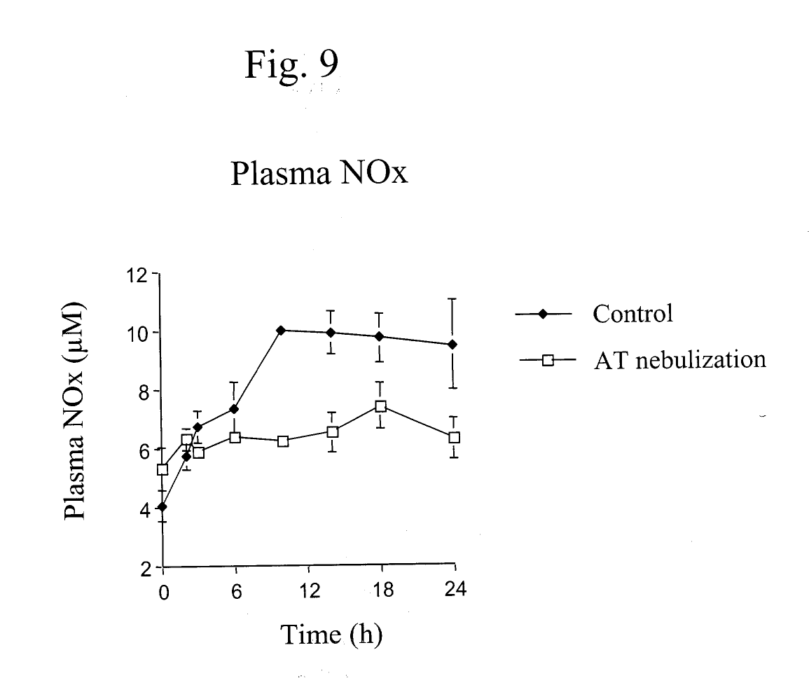

FIG. 10 is a graph depicting changes in ATIII activities in a sheep model having sepsis due to smoke inhalation.

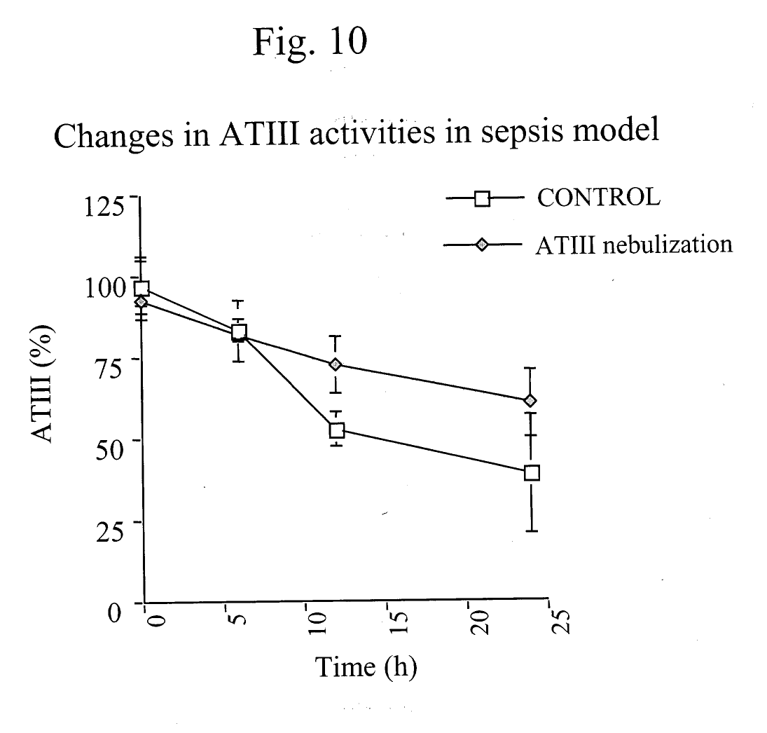

### DETAILED DESCRIPTION

It was found that the use of an aerosol form of ATIII reduced acute septic lung injury at lower doses than intravenously administered ATIII. Accordingly, the invention features aerosol formulations including ATIII, as well as, methods of using such aerosol forms of ATIII to treat a subject having a lung disorder, e.g., lung injury or inflammation.

The term “treat” or “treatment” as used herein refers to alleviating or reducing one or more symptom(s) associated with a lung disorder. For example, symptoms of lung injury and/or inflammation include: 1) reduced pulmonary gas exchange; 2) reduced pulmonary shunt fraction; 3) extracellular fibrin deposition; 4) increased vascular permeability; 5) decreased lipoprotein surfactant deposition; 6) tissue remodeling; 7) coagulation; and/or 8) increased alveolar tension. As used herein, an amount of an aerosolized form of ATIII effective to treat a lung disorder, or a “therapeutically effective amount” refers to an amount of ATIII aerosol which is effective, upon single or multiple dose administration to a subject, in curing, alleviating, relieving or improving a subject with a lung disorder as described herein beyond that expected in the absence of such treatment.

The ATIII can be administered alone, e.g., as a dry powder formulation, or with a pharmaceutically acceptable carrier. Pharmaceutically acceptable carriers include, e.g., sterile water, saline and alcohols. The pharmaceutical ATIII aerosol composition can further include other therapeutic agents (e.g., other agents which alleviate or reduce lung inflammation or injury), or other pharmaceutical adjuvants, diluents, etc. The ATIII can be administered, e.g., as a complex with, or encapsulated in a liposome.

For administration by inhalation, the compounds can be delivered in the form of an aerosol spray from pressured container or dispenser which contains a suitable propellant, e.g., a gas such as carbon dioxide, or a nebulizer. As used herein, the term “aerosols” refers to dispersions in air of solid or liquid particles, of fine enough particle size and consequent low settling velocities to have relative airborne stability (See Knight, V., Viral and Mycoplasmal Infections of the Respiratory Tract. 1973, Lea and Febiger, Phila. Pa., pp. 2).

The nebulization of ATIII may be achieved by a gas pressure or by ultrasound. Generally speaking, a nebulizer is an apparatus permitting the administration of aerosols. The nebulizers may be of any type and their structures are known to a person skilled in the art, and these devices are commercially available. The aerosols of the invention can be made by nebulizing an ATIII containing solution using a variety of known nebulizing techniques. One nebulizing system is the “wo-phase” system which consists of a solution or a suspension of active ingredient in a liquid propellant. Both liquid and vapor phases are present in a pressurized container andwhen a valve on the container is opened, liquid propellant containing the solution or suspension is released. This can result in fine aerosol mist or aerosol wet spray.

There are a variety of nebulizers that are available to produce aerosols including small volume nebulizers. Compressor driven nebulizers incorporate jet technology and use compressed air or medical oxygen to generate the aerosol. Commercially available devices are available from Healthdyne Technologies Inc; Invacare Inc.; Mountain Medical Equipment Inc.; Pari Respiratory Inc.; Mada Medical Inc.; Puritan-Bennet; Schuco Inc.; Omron Healthcare Inc.; DeVilbiss Health Care Inc; and Hospitak Inc. Ultrasonic nebulizers, e.g., an ultrasonic type nebulizer with a quartz crystal vibrating at high frequency, can also be used to deliver the ATIII.

Toxicity and therapeutic efficacy of such ATIII aerosols can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Other methods of determining the dosage of ATIII will include measuring a subject's circulating ATIII levels prior to treatment with ATIII. Based on circulating ATIII levels, the dosage of ATIII can be adjusted to be 50%, 100%, 150%, 250%, 300% greater than initial circulating levels.

The amount of aerosol formulation administered will typically in the in range of about 10 U/kg to about 250 U/kg of body weight, preferably about 25 U/kg to about 175 U/kg of body weight.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

### EXAMPLE

Recombinantly produced ATIII was dissolved in saline (42 mg/ml). Ten merino ewes were surgically prepared for the study. Five to seven days later, the animals received a tracheostomy and 48 breaths of cotton smoke (<40° C.). Pseudomonas aeruginosa was suspended in 30 mL saline, which contains 2-5×10¹¹ cfu, injected into the airway using a bronchoscope. After the bacterial challenge, the animals were ventilated mechanically with 100% O₂. Saline was used as a control. Saline (n=5) or ATIII (n=5) was nebulized (10 ml each) by an ultrasonic nebulizer at 1 hour after injury and every 4 hours thereafter throughout the 24 hour study.

Pulmonary gas exchange (PaO2/FiO2 ratio), shunt fraction, and lung wet/dry weight ratio were significantly attenuated by ATIII nebulization as shown in Table 1 below.

TABLE I

| Treatment | Saline | Antithrombin III |
|---|---|---|
| PaO2/FiO2 | 94 ± 22 | 206 ± 41* |
| Shunt fraction (%) | 45 ± 5 | 23 ± 4* |
| MAP (mmHg) | 71.3 ± 9.0 | 84.6 ± 12.0 |
| Lung Wet/Dry Ratio | 6.4 ± 0.3 | 5.4 ± 0.1* |

*p < 0.05 versus saline

Even though the total dose of ATIII was half of previously done in intravenous studies (see Murakami (2001) Am. J. Resp. Crit. Care Med. 163:A553), the outcomes were more effective than intravenous administration. No adverse effects were observed. Thus, aerosolized ATIII was beneficial in septic acute lung injury following smoke inhalation and pneumonia in sheep.

A number of embodiments of the invention have been described. Nevertheless, it will be understood that various modifications may be made without departing from the spirit and scope of the invention. Accordingly, other embodiments are within the scope of the following claims.

1. A method of treating acute lung injury in a subject, comprising:
administering by inhalation a therapeutically effective amount of antithrombin III such that the lung injury is treated;
wherein the antithrombin III is administered at a dose of about 10-300 U/kg of body weight; and,
wherein the antithrombin III is administered in the lungs at the site of the proximate acute injury.

2. The method of claim 1, wherein the lung injury is septic acute lung injury.

3. The method of claim 1, wherein the lung injury is acute respiratory distress syndrome (ARDS).

4. The method of claim 1, wherein the lung injury is in response to exposure to a viral agent.

5. The method of claim 4, wherein said viral agent is Pseudomonas pneumonia.

6. The method of claim 1, wherein the lung injury is in response to smoke or smoke and asbestos.

7. The method of claim 1, wherein the antithrombin III is administered using an ultrasonic nebulizer.

8. The method of claim 1, wherein the antithrombin III is plasma derived antithrombin III.

9. The method of claim 1, wherein the antithrombin III is recombinantly produced antithrombin III.

10. The method of claim 9, wherein the recombinantly produced antithrombin III is transgenically produced antithrombin III.

11. The method of claim 1, wherein the subject is administered more than one dose of the antithrombin III.

13. The method of claim 1, wherein the antithrombin III is administered at a dose of about 25-125 U/kg of body weight.

14. A method of treating acute lung injury in a subject, comprising:
administering by inhalation a therapeutically effective amount of antithrombin III such that the lung injury is treated; and,
wherein said lung injury is in response to the activity of Pseudomonas aeruginosa; and,
wherein the antithrombin III administered is transgenically produced antithrombin III; and
wherein the antithrombin III is administered at a dose of about 10-300 U/kg of body weight.
